(19) United States
(12) Patent Application Publication
(10) Pub. No.: US 2010/0330703 A1
(43) Pub. Date: Dec. 30, 2010

(54) ASSAYS INVOLVING COLORIMETRIC AND OTHER SIGNALING

(75) Inventors: Howard Bernstein, Cambridge, MA (US); Donald E. Chickering, III, Framingham, MA (US); Timothy M. Blicharz, Boston, MA (US)

Correspondence Address: WOLF GREENFIELD & SACKS, P.C., 600 ATLANTIC AVENUE, BOSTON, MA, 02210-2206, US

(73) Assignee: Seventh Sense Biosystems, Inc., Cambridge, MA (US)

(21) Appl. No.: 12/822,393

(22) Filed: Jun. 24, 2010

Related U.S. Application Data
(60) Provisional application No. 61/269,436, filed on Jun. 24, 2009.

(51) Int. Cl. G01N 33/543; G01N 21/00; G01N 21/62

(52) U.S. Cl. 436/518; 436/164; 436/171

(57) ABSTRACT

The present invention generally relates to particles and, in particular, to methods of determining binding involving particles, e.g., using colorimetric and other signaling techniques. In one aspect, a mixture of particles of different colors (e.g., at least a first color and a second color) is provided that exhibits a first collective color, e.g., due to the presence of the different colors of particles within the mixture. The mixture can then be exposed to a medium containing a binding partner able to preferentially bind to some of the particles, e.g., particles of a first color relative to particles of a second color. The bound particles can be separated in some fashion (e.g., filtration, gravity, magnetism, centrifugal separation, etc.), such that the mixture exhibits a second collective color, e.g., due to the presence of a greater number of particles of the second color relative to the number of particles of the first color. Accordingly, by visualizing or otherwise determining a color change, a binding event may be determined. Other aspects of the invention relate to kits involving such particles, methods of promoting the making or use of such particles, or the like.

### RELATED APPLICATIONS

This application claims the benefit of U.S. Provisional Patent Application Ser. No. 61/269,436, filed Jun. 24, 2009, entitled “Devices and Techniques Associated with Diagnostics, Therapies, Other Applications, Including Skin-Associated Applications,” by Levinson, et al., incorporated herein by reference.

### FIELD OF INVENTION

The present invention generally relates to particles and, in particular, to methods of determining binding involving particles, e.g., using colorimetric and other signaling techniques.

### BACKGROUND

Particles such as microparticles and nanoparticles have been used in a variety of applications. Typically, a “microparticle” is a particle having an average diameter on the order of micrometers (i.e., between about 1 micrometer and about 1 mm), while a “nanoparticle” is a particle having an average diameter on the order of nanometers (i.e., between about 1 nm and about 1 micrometer. Such particles are typically spherical. In addition, such particles are typically homogenous, and have the same composition throughout the particle. Given such homogeneity, uses of such particles are often limited.

### SUMMARY OF THE INVENTION

The present invention, in some aspects, generally relates to particles and, in particular, to methods of determining binding involving particles, e.g., using colorimetric and other signaling techniques. The subject matter of the present invention involves, in some cases, interrelated products, alternative solutions to a particular problem, and/or a plurality of different uses of one or more systems and/or articles.

In one aspect, the present invention is directed to a method. In one set of embodiments, the method includes acts of providing a mixture comprising, at least, particles of a first color and particles of a second color, the mixture exhibiting a first collective color; exposing the mixture of particles to a medium and allowing at least some of the particles to bind to a component of the medium via a reaction entity; separating, at least in part, bound particles from particles remaining unbound, thereby forming a collection of particles displaying a second collective color; and visualizing the second collective color as distinct from the first collective color, thereby determining the binding.

In another set of embodiments, the method includes an act of determining a characteristic of a sample by visualizing a color change caused by a change in a population of particles of at least a first color and particles of at least a second color upon preferential binding of the particles of the first color to a component of the sample via a reaction entity.

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B illustrate particles of different color contained in a fluid, and the removal of some of those particles, according to one embodiment of the present invention.

### DETAILED DESCRIPTION

The present invention generally relates to particles and, in particular, to methods of determining binding involving particles, e.g., using colorimetric and other signaling techniques. In one aspect, a mixture of particles of different colors (e.g., at least a first color and a second color) is provided that exhibits a first collective color, e.g., due to the presence of the different colors of particles within the mixture. The mixture can then be exposed to a medium containing a reaction entity able to preferentially bind to some of the particles, e.g., particles of a first color relative to particles of a second color. The bound particles can be separated in some fashion (e.g., filtration, gravity, magnetism, centrifugal separation, etc.), such that the mixture exhibits a second collective color, e.g., due to the presence of a greater number of particles of the second color relative to the number of particles of the first color. Accordingly, by visualizing or otherwise determining a color change, a binding event may be determined. Other aspects of the invention relate to kits involving such particles, methods of promoting the making or use of such particles, or the like.

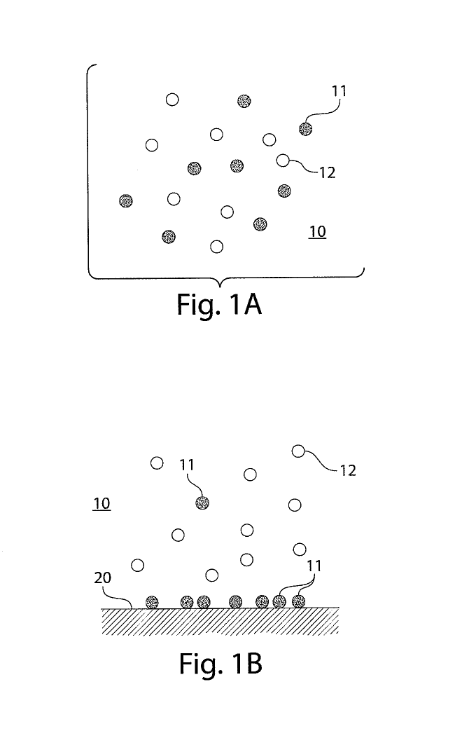

A non-limiting example is now illustrated with respect to FIG. 1. In this figure, fluid 10 contains particles of a first color 11 and particles of a second color 12. The particles may be of any suitable size, for example, microparticles or nanoparticles, and may be formed of any suitable material, e.g., a polymer. Although two colors are shown here, this is for purposes of clarity, and in other embodiments, more than two colors of particles may be present, or the two types of particles may be distinguished on the basis of visual or other features besides, or in addition to, color, for example, size or shape. The colors may be chosen to be any suitable color, for example, white and black as shown in FIG. 1A (again, for clarity), red and green, red and blue, red and yellow, blue and yellow, etc., and may be created using fluorescence, dyes, phosphorescence, or other techniques such as those described below. The collective color of fluid 10, as seen visually, may be the combination of the colors of the particles in the fluid, (e.g., particles 11 and particles 12), for example, gray in this example (a combination of black and white).

One or more types of particles may be preferentially removed from the fluid, relative to other particles in the fluid, and this removal may be determinable, e.g., visually, by a change in the collective color of the overall fluid, or by other observable properties of the fluid, e.g., a change in turbidity, a change in the amount of suspended material, a change in transparency, translucency, etc. For example, as shown in FIG. 1B, particles of the first color 11 have been removed from the fluid, leaving behind particles of second color 12 in the fluid. As shown in this figure, particles of the first color have been adsorbed onto a surface 20; in other embodiments, however, other removal techniques may be used, as discussed below. The collective color of the fluid, after removal, may preferentially be the color of the second particles (white in this example). Note that removal of particles 11 from the fluid may not necessarily be perfect; as illustrated in FIG. 1B, some particles of the first color may still remain in the fluid, although the fluid has now been enriched in the particles of the second color, relative to the particles of the first color.

Removal of the particles of the first color may be performed using a variety of techniques. For example, in one set of embodiments, the particles of the first color may include a reaction entity able to recognize an analyte. Upon exposure of the particles within the fluid to the analyte, the particles of the first color may bind via the reaction entity to the analyte, while particles of the second color do not bind to the analyte, or do not bind as preferentially as particles of the first color. Such binding may cause agglomerates to form, and/or precipitate in some cases. The agglomerates can be removed from the fluid using any suitable technique, e.g., filtration, centrifugation, or in some cases, time (e.g., by allowing the agglomerates to settle out of a fluid via gravity). Accordingly, determination of a change in color of the fluid may be used to determine whether binding to the analyte occurred or not (e.g., in an assay to determine whether the analyte was present or not), and in some cases, the amount of binding may also be determined, e.g., by observing the degree to which the color of the fluid changed.

As another example, e.g., as is shown in FIG. 1B, the fluid containing particles may be exposed to a surface that preferentially attracts particles of the first color, relative to other particles in the fluid. For instance, the particles of the first color may be formed of a material that preferentially adsorbs onto the surface, the surface may contain reaction entities able to preferentially bind to particles of the first color and/or to species present on their surfaces, the particles may become bound to the surface through an intermediate species, the particles of the first color may be magnetically susceptible and a magnet used to draw the particles towards the surface, or the like. Other methods for removing particles will be discussed in detail below. Accordingly, upon exposure of the fluid to the surface, particles of the first color 11 may become attracted to the surface, thereby leaving behind particles 12 in the fluid. This can be detected, for example, through visual observation, analytical techniques, or the like.

Accordingly, one aspect of the present invention is directed to particles contained in a fluid that are removable from the fluid. The fluid may be any fluid able to contain the particles, for example, and may include liquids, but may also include free-flowing solid particles, viscoelastic fluids, and the like. As used herein, the term “fluid” generally refers to a substance that tends to flow and to conform to the outline of its container. Typically, fluids are materials that are unable to withstand a static shear stress, and when a shear stress is applied, the fluid experiences a continuing and permanent distortion. The fluid may have any suitable viscosity that permits at least some flow of the fluid. Non-limiting examples include water and other aqueous solutions, hydrophilic liquids (e.g., ethanol), hydrophobic liquids (e.g., silicone oils, mineral oils, hydrocarbon oils, etc.), or the like.

The particles may be, for example, microparticles, nanoparticles, colloids, or the like. In some cases, a plurality of particles may be used, some or all of which particles may be substantially the same. For example, at least about 10%, at least about 30%, at least about 40%, at least about 50%, at least about 60%, at least about 70%, at least about 80%, at least about 90%, at least about 95%, or at least about 99% of the particles may have the same shape, and/or may have the same composition. For example, in one embodiment, at least about 50% of the particles delivered to the skin may have the same shape, and/or may have the same composition. For instance, at least about 50% of the particles may be anisotropic particles. In some cases, there may be more than one population of particles present in the fluid, for example, a first population of particles and a second population of particles. In other embodiments, there may be additional populations of particles presents (e.g., a third population of particles, a fourth population of particles, etc.). Each of the particles in each of the populations may substantially the same, e.g., as discussed above. However, the populations may be distinguishable in some fashion, e.g., color, shape, size, or the like. In other embodiments, there may be more than one population of particles that are each the same color, but can otherwise be distinguished, for example, due to different functional moieties on the surface, different shapes, sizes, etc.

As a non-limiting example, in one set of embodiments, the populations of particles may be distinguished on the basis of color, e.g., a first population of particles may have a first color, while a second population of particles may have a second color. The colors may be any colors that are distinguishable, e.g., visually or by the aid of an instrument, such as a fluorimeter or a colorimetric sensor. The colors may be, for example, red and green, red and blue, red and yellow, blue and yellow, green and yellow, green and blue, white and black, etc., and may be created using any suitable technique. For example, the particle may be a fluorescent particle or contain a fluorescent species. As other examples, the particles may contain dyes, pigments, phosphorescent species, or the like. In yet another example, one or more of the particles may be quantum dots.

If more than one population of particles is present in a fluid, some or all of which populations have a different color, the overall appearance of the fluid may be a color that is a blending of the colors of the various populations, and accordingly, in some cases, the composition of the particles in the fluid may be determined on that basis. For example, if particles of a first color and particles of a second color are present in a fluid, the collective color of the fluid may be a blending of the first and second colors, and this can be determined visually or by the aid of an instrument, such as a fluorimeter or a colorimetric sensor. However, if the particles of the first color are preferentially removed, relative to the other particles, then the resultant collective color of the fluid may be closer to that of the color of the particles of the second color.

The particles may be formed from any suitable material, and if more than one population of particles is present, the particles need not all be formed from the same material. The particles may be formed of any suitable material, depending on the application. For example, the particles may comprise a glass, and/or a polymer such as polyethylene, polystyrene, silicone, polyfluoroethylene, polyacrylic acid, a polyamide (e.g., nylon), polycarbonate, polysulfone, polyurethane, polybutadiene, polybutylene, polyethersulfone, polyetherimide, polyphenylene oxide, polymethylpentene, polyvinylchloride, polyvinylidene chloride, polyphthalamide, polyphenylene sulfide, polyester, polyetheretherketone, polyimide, polymethylmethacylate and/or polypropylene. In some cases, the particles may comprise a ceramic such as tricalcium phosphate, hydroxyapatite, fluorapatite, aluminum oxide, or zirconium oxide. In some cases (for example, in certain biological applications), the particles may be formed from biocompatible and/or biodegradable polymers such as polylactic and/or polyglycolic acids, polyanhydride, polycaprolactone, polyethylene oxide, polyacrylamide, polyacrylic acid, polybutylene terephthalate, starch, cellulose, chitosan, and/or combinations of these. In one set of embodiments, the particles may comprise a hydrogel, such as agarose, collagen, or fibrin. The particles may include a magnetically susceptible material in some cases, e.g., a material displaying paramagnetism or ferromagnetism. For instance, the particles may include iron, iron oxide, magnetite, hematite, or some other compound containing iron, or the like. In another embodiment, the particles can include a conductive material (e.g., a metal such as titanium, copper, platinum, silver, gold, tantalum, palladium, rhodium, etc.), or a semiconductive material (e.g., silicon, germanium, CdSe, CdS, etc.). Other particles potentially useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, or GaAs. The particles may include other species as well, such as cells, biochemical species such as nucleic acids (e.g., RNA, DNA, PNA, etc.), proteins, peptides, enzymes, nanoparticles, quantum dots, fragrances, indicators, dyes, fluorescent species, chemicals, small molecules (e.g., having a molecular weight of less than about 1 kDa), or the like.

The particles also may be spherical or non-spherical, in some cases. For example, in one set of embodiments, populations of particles may be distinguished on the basis of shape and/or size. The particles may be, for example, oblong or elongated, or have other shapes such as those disclosed in U.S. patent application Ser. No. 11/851,974, filed Sep. 7, 2007, entitled “Engineering Shape of Polymeric Micro- and Nanoparticles,” by S. Mitragotri, et al.; International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008; U.S. patent application Ser. No. 11/272,194, filed Nov. 10, 2005, entitled “Multi-phasic Nanoparticles,” by J. Lahann, et al., published as U.S. Patent Application Publication No. 2006/0201390 on Sep. 14, 2006; or U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Patent Application Publication No. 2007/0237800 on Oct. 11, 2007, each of which is incorporated herein by reference.

In some embodiments of the invention, the fluid containing the particles itself may also have a color, although in other embodiments, the fluid may be one that is not colored (for example, fluids such as pure water, mineral oil, etc.). For example, in one set of embodiments, the fluid may contain a dye, a fluorescent entity dissolved or suspended in the fluid, or the like, which imparts a color to the fluid. This color may be the same or different from particles contained within the solution that have a color. In another set of embodiments, the fluid may contain other colored particles that do not participate in any reactions, but are used to impart a color to the fluid. In some cases, a reaction may be determined using such a system by determining a change in color of the fluid (e.g., due to agglomeration and/or precipitation of particles from the fluid). For instance, a fluid may contain particles having a first color, while the fluid itself may be a second color. Thus, the fluid containing the particles may have a collective color that is a combination of these colors. Upon binding of an analyte to a reaction entity on the surface of at least some of the particles, the particles may agglomerate and/or precipitate, or the particles may be filtered or otherwise separated as discussed herein, thereby causing the fluid to at least become enriched with respect to the second color. By detecting such changes in color in the fluid, binding of an analyte to a reaction entity, or other reactions as discussed herein, may be determined. As yet another example, a fluid may contain a first population of particles having a first color, a second population of particles having a second color, and the fluid itself may be a third color; in other embodiments, additional populations of particles may also be present.

The particles may also have any shape or size. For instance, the particles may have an average diameter of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. As discussed, the particles may be spherical or non-spherical. The average diameter of a non-spherical particle is the diameter of a perfect sphere having the same volume as the non-spherical particle. If the particle is non-spherical, the particle may have a shape of, for instance, an ellipsoid, a cube, a fiber, a tube, a rod, or an irregular shape. In some cases, the particles may be hollow or porous. Other shapes are also possible, for instance, core/shell structures (e.g., having different compositions), rectangular disks, high aspect ratio rectangular disks, high aspect ratio rods, worms, oblate ellipses, prolate ellipses, elliptical disks, UFOs, circular disks, barrels, bullets, pills, pulleys, biconvex lenses, ribbons, ravioli, flat pills, bicones, diamond disks, emarginate disks, elongated hexagonal disks, tacos, wrinkled prolate ellipsoids, wrinkled oblate ellipsoids, porous ellipsoid disks, and the like. See, e.g., International Patent Application No. PCT/US2007/077889, filed Sep. 7, 2007, entitled “Engineering Shape of Polymeric Micro- and Nanoparticles,” by S. Mitragotri, et al., published as WO 2008/031035 on Mar. 13, 2008, incorporated herein by reference.

In one set of embodiments, the particle may be an anisotropic particle, i.e., one that is not spherically symmetric (although the particle may still exhibit various symmetries). The asymmetry can be asymmetry of shape, of composition, or both. As an example, a particle having the shape of an egg or an American football is not perfectly spherical, and thus exhibits anisotropy. As another example, a sphere painted such that exactly one half is red and one half is blue (or otherwise presents different surface characteristics on different sides) is also anisotropic, as it is not perfectly spherically symmetric, although it would still exhibit at least one axis of symmetry. In one set of embodiments, at least some of the particles used to determine an analyte are anisotropic particles (in other cases, however, the particles are not necessarily anisotropic), and in some cases, substantially all of the particles are anisotropic particles. In certain cases, at least about 10%, at least about 30%, at least about 40%, at least about 50%, at least about 60%, at least about 70%, at least about 80%, at least about 90%, at least about 95%, or at least about 99% of the particles are anisotropic particles.

A particle may be anisotropic due to its shape and/or due to two or more regions that are present on the surface of and/or within the particle. For instance, the particle may include a first surface region and a second surface region that is distinct from the first region in some way, e.g., due to coloration, surface coating, the presence of one or more reaction entities, etc. The particle may include different regions only on its surface or the particle may internally include two or more different regions, portions of which extend to the surface of the particle. The regions may have the same or different shapes, and be distributed in any pattern on the surface of the particle. For instance, the regions may divide the particle into two hemispheres, such that each hemisphere has the same shape and/or the same surface area, or the regions may be distributed in more complex arrangements.

Non-limiting examples of anisotropic particles can be seen in U.S. patent application Ser. No. 11/272,194, filed Nov. 10, 2005, entitled “Multi-phasic Nanoparticles,” by J. Lahann, et al., published as U.S. Patent Application Publication No. 2006/0201390 on Sep. 14, 2006; or U.S. patent application Ser. No. 11/763,842, filed Jun. 15, 2007, entitled “Multi-Phasic Bioadhesive Nan-Objects as Biofunctional Elements in Drug Delivery Systems,” by J. Lahann, published as U.S. Patent Application Publication No. 2007/0237800 on Oct. 11, 2007, each of which is incorporated herein by reference.

The separation of particles in a fluid may be performed using any suitable technique, according to certain aspects of the invention. For example, in one embodiment, binding of an analyte to a reaction entity on the surface of at least some of the particles may cause those particles to agglomerate and/or precipitate, depending on the particular application. As a specific example, particles of a first color may bind to the analyte (and may settle out of solution, or be filtered out, etc.), while the particles of the second color may not bind to the analyte (or binds less preferentially than particles of the first color). In such situations, the fluid may appear to change color as binding occurs, and the analyte can accordingly be determined by its effects on the particles within the fluid. For example, a change in the collective color of the fluid may be the result of particles of one color preferentially binding to the analyte, relative to other particles in solution, and in some cases, the amount of color change may be quantified to determine the amount or concentration of analyte that is present.

In one set of embodiments, a population of particles (or agglomerates of particles) may be drawn to a surface and thereby separated from the fluid. Any suitable technique can be used to draw the particles to the surface. For example, reaction entities may be present on the surface of a substrate, and the population of particles may be attracted to the surface and be removed from the fluid. As another example, the particles may be drawn to the surface under the influence of various forces such as electrical forces, magnetic forces, sedimentation (gravity), centrifugal forces, or the like. For instance, the particles may be of different average dimensions, and can easily be separated on the basis of the sizes or weights (e.g., using filtration or sedimentation), or at least some of the particles may be magnetic, or at least magnetically susceptible, and a magnet used to effect separation of the particles. As other examples of separation, a population of particles may be removed from the solution by filtration (e.g., if populations of particles in the fluid have different sizes or diameters), buoyancy or density effects, or the like.

In another set of embodiments, the population of particles (or agglomerates of particles) may be separated from the fluid using filtration techniques. For example, a fluid containing particles may be passed through a filter or a membrane such that the fluid is able to pass through the membrane, while agglomerates of particles are not able to pass therethrough. In some cases, the porosity of the filter may be such that individual particles can pass through the membrane, while larger agglomerates are not able to pass through the membrane, and thus can be separated from the fluid. The filter may have, in some cases, a pore spacing to allow single particles or small aggregates of particles to pass through, while preventing larger agglomerates of particles from passing through. For example, the size of the pores within the filter may be about the same size as the average diameter of the particles, or possibly a little larger. For example, the pores may have an average or characteristic dimension of than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. The filter may be formed out of any suitable material, for example, the filter may be formed out of a polymer such as nitroceloulose, acetate, polytetrafluoroetehylene nylon, polycarbonate, polyethersulfone, polyvinylidene fluoride, polypropylene or the like. In other embodiments, the filter may be formed out of non-polymeric materials, such as silica or a ceramic material. In some cases, pressure may be used to assist in passing the fluid through the filter. In other cases, fluid may be urged through the filter through gravity, centrifugal force, or other suitable techniques.

The particles may be used for a variety of purposes. For instance, the particles may contain a reaction entity able to interact with and/or associate with an analyte, or another reaction entity, or other particles. Such particles may be useful, for example, to determine one or more analytes. In one set of embodiments, a particle may include one or more reaction entities present on the surface (or at least a portion of the surface) of the particle. The reaction entity may be any entity able to interact with and/or associated with an analyte, or another reaction entity. For instance, the reaction entity may be a binding partner able to bind an analyte. For example, the reaction entity may be a molecule that can undergo binding with a particular analyte. The reaction entities may be used, for example, to determine pH or metal ions, proteins, nucleic acids (e.g. DNA, RNA, etc.), drugs, sugars (e.g., glucose), hormones (e.g., estradiol, estrone, progesterone, progestin, testosterone, androstenedione, etc.), carbohydrates, or other analytes of interest. It should also be noted that the analyte need not be present in solution. For instance, as discussed below, an analyte may be present on a surface, and the particles may be able to orient themselves relative to the surface due to the presence of the reaction entities.

The term “binding partner” refers to a molecule that can undergo binding with a particular molecule, e.g., an analyte. For example, the binding may be highly specific and/or non-covalent. Binding partners which form highly specific, non-covalent, physiochemical interactions with one another are defined herein as “complementary.” Biological binding partners are examples. For example, Protein A is a binding partner of the biological molecule IgG, and vice versa. Other non-limiting examples include nucleic acid-nucleic acid binding, nucleic acid-protein binding, protein-protein binding, enzyme-substrate binding, receptor-ligand binding, receptor-hormone binding, antibody-antigen binding, etc. Binding partners include specific, semi-specific, and non-specific binding partners as known to those of ordinary skill in the art. For example, Protein A is usually regarded as a “non-specific” or semi-specific binder. As another example, the particles may contain an enzyme such as glucose oxidase or glucose 1-dehydrogenase, or a lectin such as concanavalin A that is able to bind to glucose.

As additional examples, binding partners may include antibody/antigen pairs, ligand/receptor pairs, enzyme/substrate pairs and complementary nucleic acids or aptamers. Examples of suitable epitopes which may be used for antibody/antigen binding pairs include, but are not limited to, HA, FLAG, c-Myc, glutatione-S-transferase, His₆, GFP, DIG, biotin and avidin. Antibodies may be monoclonal or polyclonal. Suitable antibodies for use as binding partners include antigen-binding fragments, including separate heavy chains, light chains Fab, Fab′, F(ab′)₂, Fabc, and Fv. Antibodies also include bispecific or bifunctional antibodies. Exemplary binding partners include biotin/avidin, biotin/streptavidin, biotin/neutravidin and glutathione-S-transferase/glutathione.

The term “binding” generally refers to the interaction between a corresponding pair of molecules or surfaces that exhibit mutual affinity or binding capacity, typically due to specific or non-specific binding or interaction, including, but not limited to, biochemical, physiological, and/or chemical interactions. The binding may be between biological molecules, including proteins, nucleic acids, glycoproteins, carbohydrates, hormones, or the like. Specific non-limiting examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, virus/cell surface receptor, etc. As another example, the binding agent may be a chelating agent (e.g., ethylenediaminetetraacetic acid) or an ion selective polymer (e.g., a block copolymer such as poly(carbonate-b-dimethylsiloxane), a crown ether, or the like). In some cases, binding may be between non-biological molecules, for example, between a catalyst and its substrate. As another example, the binding partners may be biotin and streptavidin, or the binding partners may be various antibodies raised against a protein.

Accordingly, certain embodiments of the invention are generally directed to assays that can be well controlled, e.g., such that their selectivity, sensitivity, dynamic range, stability, biocompatability, etc. can be controlled. For instance, a colorimetric assay involving a color change may be controlled by controlling the size of the particles, the colors of the particles, the concentration and/or location of binding partners on the surfaces of the particles, the shape or anisotropy of the particles, etc. In addition, in some embodiments, the invention is directed to a homogeneous assay. Such assays typically do not require any preparation steps, e.g., separation, washing, blocking, etc. In some cases, the assay may be determined without applying any energy and/or external chemicals to the assay, and in some cases, the assay may be determined without the use of any equipment.

For example, in one set of embodiments, the particles may contain a reaction entity able to determine an analyte. In some cases, the analyte is one that may be obtained from a subject, e.g., through withdraw of blood or interstitial fluid from the sample. One example of such an analyte is glucose (e.g., for diabetics); other potentially suitable analytes include ions such as sodium, potassium, chloride, calcium, magnesium, and/or bicarbonate; gases such as carbon dioxide or oxygen; pH; metabolites such as urea, blood urea nitrogen or creatinine; hormones such as estradiol, estrone, progesterone, progestin, testosterone, androstenedione, etc. (e.g., to determine pregnancy, illicit drug use, or the like); or cholesterol. Still other potentially suitable analytes include various pathogens such as bacteria or viruses, and/or markers produced by such pathogens. For example, a particle may include an antibody directed at a marker produced by bacteria. In addition, more than one analyte may be determined in a sample, e.g., through the use of different particle types and/or through the use of particles able to determine more than one analyte, such as those discussed above. For instance, a first set of particles may determine a first analyte and a second set of particles may determine a second analyte.

Binding partners to these and/or other species are well-known in the art. Non-limiting examples include pH-sensitive entities such as phenol red, bromothymol blue, chlorophenol red, fluorescein, HPTS, 5(6)-carboxy-2′,7′-dimethoxyfluorescein SNARF, and phenothalein; entities sensitive to calcium such as Fura-2 and Indo-1; entities sensitive to chloride such as 6-methoxy-N-(3-sulfopropyl)-quinolinim and lucigenin; entities sensitive to nitric oxide such as 4-amino-5-methylamino-2′,7′-difluorofluorescein; entities sensitive to dissolved oxygen such as tris(4,4′-diphenyl-2,2′-bipyridine) ruthenium (II) chloride pentahydrate; entities sensitive to dissolved CO₂; entities sensitive to fatty acids, such as BODIPY 530-labeled glycerophosphoethanolamine; entities sensitive to proteins such as 4-amino-4′-benzamidostilbene-2-2′-disulfonic acid (sensitive to serum albumin), X-Gal or NBT/BCIP (sensitive to certain enzymes), Tb³⁺ from TbCl₃ (sensitive to certain calcium-binding proteins), BODIPY FL phallacidin (sensitive to actin), or BOCILLIN FL (sensitive to certain penicillin-binding proteins); entities sensitive to concentration of glucose, lactose or other components, or entities sensitive to proteases, lactates or other metabolic byproducts, entities sensitive to proteins, antibodies, or other cellular products.

The term “specifically binds,” when referring to a binding partner (e.g., protein, nucleic acid, antibody, etc.), refers to a reaction that is determinative of the presence and/or identity of one or other member of the binding pair in a mixture of heterogeneous molecules (e.g., proteins and other biologics). Thus, for example, in the case of a receptor/ligand binding pair, the ligand would specifically and/or preferentially select its receptor from a complex mixture of molecules, or vice versa. An enzyme would specifically bind to its substrate, a nucleic acid would specifically bind to its complement, an antibody would specifically bind to its antigen, etc. The binding may be by one or more of a variety of mechanisms including, but not limited to ionic interactions or electrostatic interactions, covalent interactions, hydrophobic interactions, van der Waals interactions, etc.

Thus, the invention provides, in certain embodiments, particles such as anisotropic particles that are able to bind to an analyte, e.g., via a binding partner to the analyte, and such particles can be used to determine the analyte. “Determine,” in this context, generally refers to the analysis of a species, for example, quantitatively or qualitatively, and/or the detection of the presence or absence of the species. “Determining” may also refer to the analysis of an interaction between two or more species, for example, quantitatively or qualitatively, and/or by detecting the presence or absence of the interaction, e.g. determination of the binding between two species. “Determining” also means detecting or quantifying interaction between species. As an example, an analyte may cause a determinable change in a property of the particles, e.g., a change in a chemical property of the particles, a change in the appearance and/or optical properties of the particles, a change in the temperature of the particles, a change in an electrical property of the particles, etc. In some cases, the change may be one that is determinable by a human, unaided by any equipment that may be directly applied to the human. For instance, the determinable change may be a change in appearance (e.g., color), a change in temperature, the production of an odor, etc., which can be determined by a human without the use of any equipment (e.g., using the eyes).

In one set of embodiments, more than one particle may be able to bind an analyte, and/or more than one analyte may bind to a particle. In some cases, such multiple binding properties may result in the clustering of more than one particle to an analyte and/or more than one analyte to a particle. Such clustering can be determined in some fashion, e.g., via a change in color or an optical property. For instance, multiple particles, when clustered around an analyte, may become visible, e.g., as discrete aggregates and/or as a change in color. In another example, the clusters themselves may not be visible, but an optical property of the medium containing the clusters may be altered in some fashion (e.g., exhibiting different light scattering properties, different opacities, different degrees of transparency, etc.), which can be determined or correlated to determine the analyte.

As an example, an aggregate of particles may form in the absence of analyte, but disaggregate (at least partially) in the presence of the analyte, e.g., if the analyte and the particles exhibit competitive or non-competitive inhibition. Such binding and/or aggregation may be equilibrium-based in some cases, i.e., the binding and/or aggregation occurs in equilibrium with unbinding or disaggregation processes. Thus, when the environment surrounding the particles is altered in some fashion (e.g., a change in concentration of an analyte), the equilibrium may shift in response, which can be readily determined (e.g., as a change in color). It should be noted that such equilibrium-based systems may be able to determine such changes in environment, in some cases, without the need to apply any energy to determine the environmental change. In another example, aggregation may cause a change in an electrical or a magnetic property.

In addition, it should be noted that the particles, in some embodiments, may contain reaction entities that are not necessarily binding partners to an analyte. For instance, there may be first particles containing a first reaction entity and a second reaction entity that reacts with the first reaction entity; when the particles are brought together in some fashion (e.g., by exposure to an analyte or other chemical that is recognized by binding partners on each of the particles, by the application of an electrical, magnetic, and/or a mechanical force to bring the particles closer together, etc.), the first and second reaction entities may react. As a specific example, the reaction between the first and second reaction entities may be an endothermic or an exothermic reaction; thus, when the particles are brought together, a temperature change is produced, which can be determined in some fashion. As another example, a reaction between the first and second reactants may cause the release of a material.

Determination of the particles, and their separation may be performed using any suitable technique, according to another aspect of the invention. In some cases, the detection may be determined as a change in color or other suitable property of the fluid containing the particles, e.g., turbidity, translucency, transparency or opacity, viscosity, laser light scattering, density, lightening or darkening of the fluid, or the like, and in some cases, such determinations may be made by a human. If a change in color is present, it may be of any suitable change, e.g., red to blue, red to yellow, red to green, etc. Accordingly, in one set of embodiments, the change in the fluid is one that is visually detectable by a human, e.g., without the use of any laboratory equipment. For example, changes in color, turbidity, shading (e.g., darkening or lightening), transparency, translucency, or the like may be determined by a human. Typically, such detection is made unaided by any equipment. As a specific non-limiting example, a fluid containing particles having various colors may be exposed to a sample suspected of containing an analyte; visual observation of the fluid upon addition of the sample to the fluid (e.g., a change in color) may be used to determine whether the sample contains the analyte or not. In other embodiments, however, such determinations may be aided by suitable equipment, e.g., using fluorimeters, microplate readers, CCD cameras, colorimeters, photomultiplier tubes, photodiodes, laser light scattering, or the like.

As an example, an optical property of the medium containing the clusters may be altered in some fashion (e.g., exhibiting different light scattering properties, different opacities, different degrees of transparency, etc.), which can be correlated with the analyte. In some cases, the color may change in intensity, for example, the clustering of particles may bring two or more reactants into close proximity. For example, in one set of embodiments, the medium may contain two different particles, which are distinguishable in some fashion, for example, with respect to color, shape, size, etc., as discussed herein. For instance, a solution may contain a first particle having a first functionality, and a second particle having a second functionality different from the first functionality. The particles may be the same or different colors. In the presence of an analyte, the two types of particles may agglomerate or precipitate, for example, due to a reaction catalyzed by the analyte or due to interactions of the particles with the analyte (e.g., if the surface of the particles contains binding partners to the analyte). Upon agglomeration, the color of the solution may change due to the agglomeration of the particles.

Other properties may also be determined besides color. Accordingly, it should be understood that the use of “color” with respect to particles as used herein is by way of example only, and other properties may be determined instead of or in addition to color. For instance, clustering of aniostropic particles may cause a change in an electrical or a magnetic property of the particles, which can be determined by determining an electrical or a magnetic field. As another example, the first region and the second region may have different reactivities (e.g., the first region may be reactive to an enyzme, an antibody, etc.), and aggregation of the particles may cause a net change in the reactivity. As still another example, size may be used to determine the particles and/or the analyte. For instance, the aggregates may be visually identifiable, the aggregates may form a precipitant, or the like. Thus, for example, the particles (which may be anisotropic or not anisotropic) may appear to be a first color when separate, and a second color when aggregation occurs. In some cases, an assay (e.g., an agglutination assay) may be used to determine the aggregation. In another set of embodiments, an ordering of the particles may be determined. For example, in the absence of an analyte, the particles may be ordered on the surface of a substrate; while in the presence of an analyte, the particles may bind to the analyte and become disordered relative to the surface. This ordering may be determined, for example, as a change in an optical property of the surface (e.g., index of refraction, color, transparency or opacity, etc.). It should also be understood that a change in color may be identified as a change in hue (e.g., from red to yellow, from orange to green, etc.), a change in shade (e.g., from yellow to brown, from light blue to dark blue, etc.), a change in intensity, or the like.

In another aspect, the present invention is directed to a kit including one or more of the compositions previously discussed, e.g., a kit including particles. A “kit,” as used herein, typically defines a package or an assembly including one or more of the compositions of the invention, and/or other compositions associated with the invention, for example, as previously described. Each of the compositions of the kit may be provided in liquid form (e.g., in solution), or in solid form (e.g., a dried powder). In certain cases, some of the compositions may be constitutable or otherwise processable (e.g., to an active form), for example, by the addition of a suitable solvent or other species, which may or may not be provided with the kit. Examples of other compositions or components associated with the invention include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, tapes, adhesives, and the like, for example, for using, administering, modifying, assembling, storing, packaging, preparing, mixing, diluting, and/or preserving the compositions components for a particular use, for example, to a sample and/or a subject.

A kit of the invention may, in some cases, include instructions in any form that are provided in connection with the compositions of the invention in such a manner that one of ordinary skill in the art would recognize that the instructions are to be associated with the compositions of the invention. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In some embodiments, the present invention is directed to methods of promoting one or more embodiments of the invention as discussed herein, for example, methods of promoting the making or use of particles such as those discussed above, methods of promoting kits as discussed above, or the like. As used herein, “promoted” includes all methods of doing business including, but not limited to, methods of selling, advertising, assigning, licensing, contracting, instructing, educating, researching, importing, exporting, negotiating, financing, loaning, trading, vending, reselling, distributing, repairing, replacing, insuring, suing, patenting, or the like that are associated with the systems, devices, apparatuses, articles, methods, compositions, kits, etc. of the invention as discussed herein. Methods of promotion can be performed by any party including, but not limited to, personal parties, businesses (public or private), partnerships, corporations, trusts, contractual or sub-contractual agencies, educational institutions such as colleges and universities, research institutions, hospitals or other clinical institutions, governmental agencies, etc. Promotional activities may include communications of any form (e.g., written, oral, and/or electronic communications, such as, but not limited to, e-mail, telephonic, Internet, Web-based, etc.) that are clearly associated with the invention.

In one set of embodiments, the method of promotion may involve one or more instructions. As used herein, “instructions” can define a component of instructional utility (e.g., directions, guides, warnings, labels, notes, FAQs or “frequently asked questions,” etc.), and typically involve written instructions on or associated with the invention and/or with the packaging of the invention. Instructions can also include instructional communications in any form (e.g., oral, electronic, audible, digital, optical, visual, etc.), provided in any manner such that a user will clearly recognize that the instructions are to be associated with the invention, e.g., as discussed herein.

U.S. Provisional Patent Application Ser. No. 61/163,793, filed Mar. 26, 2009, entitled “Compositions and Methods for Diagnostics, Therapies, and Other Applications,” by Levinson is incorporated herein by reference. Also incorporated herein by reference are U.S. Provisional Patent Application Ser. No. 61/058,796, filed Jun. 4, 2008, entitled “Compositions and Methods for Diagnostics, Therapies, and Other Applications,” by Levinson; U.S. Provisional Patent Application Ser. No. 61/269,436, filed Jun. 24, 2009, entitled “Devices and Techniques Associated with Diagnostics, Therapies, Other Applications, Including Skin-Associated Applications,” by Levinson, et al.; U.S. patent application Ser. No. 12/716,233, filed Mar. 2, 2010, entitled “Systems and Methods for Creating and Using Suction Blisters or Other Pooled Regions of Fluid within the Skin,” by Levinson, et al.; U.S. patent application Ser. No. 12/716,229, filed Mar. 2, 2010, entitled “Devices and Techniques Associated with Diagnostics, Therapies, and Other Applications, Including Skin-Associated Applications,” by Bernstein, et al.; and U.S. patent application Ser. No. 12/716,226, entitled “Techniques and Devices Associated with Blood Sampling,” by Levinson, et al.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
providing a mixture comprising, at least, particles of a first color and particles of a second color, the mixture exhibiting a first collective color;
exposing the mixture of particles to a medium and allowing at least some of the particles to bind to a component of the medium via a reaction entity;
separating, at least in part, bound particles from particles remaining unbound, thereby forming a collection of particles displaying a second collective color; and
visualizing the second collective color as distinct from the first collective color, thereby determining the binding.

2. The method of claim 1, wherein the bound particles are drawn to a surface.

3. The method of claim 1, wherein the bound particles are drawn to a surface using magnetism.

4. The method of claim 1, wherein the bound particles are drawn to a surface using centrifugation.

5. The method of claim 1, wherein the bound particles are drawn to a surface using gravity.

6. The method of claim 1, wherein the component of the medium is a surface, and the bound particles are particles bound to the surface.

7. The method of claim 1, wherein the reaction entity is an antibody.

8. The method of claim 1, wherein the bound particles form an agglomerate within the medium.

9. The method of claim 1, wherein separation comprises passing the mixture through a filter.

10. The method of claim 1, wherein the mixture is a fluid.

11. The method of claim 10, wherein the fluid is a liquid.

12. The method of claim 1, wherein the fluid is colored.

13. The method of claim 1, wherein the particles of the first color have a first functionality, and the particles of the second color have a second functionality distinguishable from the first functionality.

14. The method of claim 1, wherein the visualizing is performed using the naked eye.

15. The method of claim 1, wherein the visualizing is performed spectroscopically.

16. The method of claim 1, wherein the particles of the first color have an average diameter of less than about 5 mm.

17. The method of claim 1, wherein the first color and the second color are substantially the same.

18. A method comprising:
determining a characteristic of a sample by visualizing a color change caused by a change in a population of particles of at least a first color and particles of at least a second color upon preferential binding of the particles of the first color to a component of the sample via a reaction entity.

19. The method of claim 18, wherein the reaction entity is bound to a surface.

20. The method of claim 18, wherein the sample is a liquid.

21. The method of claim 18, wherein the reaction entity is an antibody.

22. The method of claim 18, wherein the particles of the first color have a first functionality, and the particles of the second color have a second functionality distinguishable from the first functionality.

23. The method of claim 18, wherein the visualizing is performed using the naked eye.

24. The method of claim 18, wherein the particles of the first color have an average diameter of less than about 5 mm.

25. The method of claim 18, wherein the first color and the second color are substantially the same.
